(19) United States
(12) Patent Application Publication
(10) Pub. No.: US 2013/0035602 A1
(43) Pub. Date: Feb. 7, 2013

(54) MOBILE REMOTE ALCOHOL MONOTORING DEVICE

(71) Applicant: Gil Gemer, Woodbury, MN (US)

(72) Inventor: Gil Gemer, Woodbury, MN (US)

(73) Assignee: 3M Innovative Properties Company

(21) Appl. No.: 13/198,552

(22) Filed: Aug. 4, 2011

(51) Int. Cl. A61B 5/0205

(52) U.S. Cl. 600/484

(57) ABSTRACT

A monitoring apparatus including an ethanol level sensor; an ECG sensor for generating an ECG circuit for sampling an electrical activity of a heart of a monitored person being monitored; and an air inlet component connected to an air flow sensor; wherein a first electrode is attached to the breath inlet component and wherein the ECG circuit is generated when a monitored person is in electrical contact with the first electrode and the second electrode of the ECG.

### FIELD OF THE INVENTION

The present disclosure relates to breath alcohol monitoring devices in general, and in particular to a mobile breath alcohol monitoring device.

### BACKGROUND

Persons may be monitored for their blood alcohol level in a variety of situations. For example, a person placed under alcohol supervision as an alternative to incarceration may be required to have their blood alcohol level tested at regular or random times. Currently, these individuals are monitored with home installed alcohol monitoring devices. Two types of home installed alcohol monitoring devices are known in the art. In the first type of device, the monitored person is required to provide a breath sample by blowing into a stationary alcohol monitoring device at random times. An ethanol level sensor in the device tests the breath sample for alcohol content to estimate the monitored person's blood alcohol level. And then, if the blood alcohol level exceeds a given level, an alarm may be issued or a report may be generated and may be sent to a remote device or location. Such stationary alcohol monitoring devices are typically located in a fixed place, such as the home of the monitored person. Therefore, the blood alcohol level of the monitored person typically can only be estimated from a breath sample when the person is in the home environment.

In the second type of home installed alcohol monitoring device, a person monitored for their blood alcohol level may also be required to wear an ankle bracelet for the duration of a court-ordered abstinence period. Current bracelets periodically capture alcohol level readings by sampling perspiration that evaporates into the air above the skin before it is perceived as moisture on the skin, but can still be detected and sampled, known as insensible perspiration. An ethanol level sensor in the bracelet tests the insensible perspiration to estimate the monitored person's blood alcohol level. The bracelet stores the test data related to blood alcohol level and, at pre-determined intervals, transmits the data via radio-frequency (RF) signal to a base station in the home that are received only when the monitored person is within range of the base station. Similar to the first type of home installed alcohol monitoring device, if the blood alcohol level exceeds a given level an alarm may be issued or a report may be generated and may be sent to a remote device or location.

There is a need for an improved alcohol monitoring device, preferably one that overcomes the limitations of existing alcohol monitoring devices.

### SUMMARY

One technical problem dealt with by the present disclosure is remotely monitoring a monitored person's blood alcohol level when the monitored person is away from a place where a stationary device for blood alcohol measurement is located. A monitored person may be monitored according to a court order. When using a fixed alcohol remote monitoring device located at one place, for example, the home of the monitored person, no monitoring can be conducted when the monitored person is away from the home or from another location where the mobile alcohol remote monitoring device is located. Thus, the monitored person may consume alcohol shortly after leaving a monitored location, such as the home, to go to a location without a fixed monitoring device, such as at work, and return later when his blood alcohol level is under the allowed threshold. Methods known in the art, such as a bracelet, periodically capture alcohol readings by sampling the insensible perspiration collected from the air above the skin, may not be accurate and may not provide adequate proof in court. Such methods rely on the monitored person not removing the bracelet and do not include positive identification devices.

Another technical problem dealt with by the present disclosure is identifying the monitored person by the mobile alcohol remote monitoring device. A person who is remotely monitored may let another person's breath alcohol level be tested, resulting in incorrect correlation of the results with the relevant monitored person.

Yet, another technical problem dealt with by the present disclosure is monitoring the location of the monitored person, in order, for example, to invoke an alarm when the monitored person is located in an area that is forbidden for this monitored person. Such an area may be, for example, a pub.

One technical solution includes a mobile alcohol remote monitoring device that can monitor the monitored person at any location, and at any time, by performing a breath alcohol test. Breath alcohol testing can be more accurate than the method of sampling the insensible perspiration collected from the air above the skin that is used in portable monitoring devices. Such a mobile alcohol remote monitoring device may ensure the monitored person's identity by conducting a biometric or a biological verification at the time of the breath alcohol testing.

In some exemplary embodiments, the mobile alcohol remote monitoring device includes an authentication device. In some exemplary embodiments, an authentication device may be an electrocardiogram (ECG) monitor for verifying the identity of the monitored person. Verification may be done by comparing the captured ECG signals with a reference ECG signals of the monitored person. The comparing may be performed in the mobile alcohol remote monitoring device, or in a remote computerized device, such as for example a remote monitoring station, a home monitoring unit, a relay unit and the like. In some embodiments of the present disclosure, the reference ECG signal, the captured ECG signal, or the results of the comparison between the reference ECG signal and a captured ECG signal may be sent to the remote computerized device. The reference ECG signal may be stored in the mobile alcohol remote monitoring device or in the exemplary device remote to the mobile alcohol remote monitoring device, such as a remote monitoring station, a home monitoring unit, a relay unit and the like. The ECG monitor may be activated upon breathing by closing an electric circuit between an electrode that is placed in a position such that when the mobile remote alcohol monitoring device is used the first electrode is in contact with the lips of the monitored person, and a second electrode is in contact with a limb or other body part of the monitored person, such as the palm of the hand. Such an electrical circuit may be generated automatically while the monitored person holds the mobile alcohol remote monitoring device or is in contact with the mobile alcohol remote monitoring device, and breathes into the mobile remote alcohol monitoring device.

In some exemplary embodiments, the authentication device may be an image capturing device such as a camera, and/or other identification devices such as a fingerprint capturing device, an iris recognition device, a biometric recognition device, and the like. An actuating device may be mounted on the mobile alcohol remote monitoring device so as to assist the monitored person to position her or his face while blowing into a straw connected to the mobile alcohol remote monitoring device. Positioning the monitored person's face correctly can result in a properly focused, centered identifying image of consistent scale and preferably devoid of background. The image may be taken automatically and then either compared to a reference image that is stored in the mobile alcohol remote monitoring device or alternatively an analysis may be performed on the captured image to compare facial characteristics, such as for example, geometric characteristics of the face (distance between pupils, length of lips, width of nose, etc.) and compare these features between the captured and stored images. Alternatively, a remote computerized device may store a reference image, and the image taken may be sent to the remote computerized device for performing the comparison. In some other embodiments the image may be compared partially, such that only some features are compared, and a decision is made based on the feature comparison at the mobile alcohol remote monitoring or at the remote computerized device. Such decision can be that the image taken is that of the monitored person, that the image taken is not of the monitored person or that the image taken resembles the monitored person by a certain percentage, probability or likelihood.

In some exemplary embodiments, the authentication device may be activated as a result of the monitored person blowing into the straw, thus ensuring that the image taken, assessed and authenticated is of the monitored person blowing into the mobile remote alcohol monitoring device. When the authentication device is an ECG, it may be activated when a first electrode of the ECG device comes into contact with the lips of the monitored person while blowing into the straw and a second electrode contacts the monitored person's body or limb and the signal passes through the heart. The first electrode of the ECG device may be attached to the straw to cause the first electrode to come into contact with the lips of the monitored person while blowing into the straw. A second electrode may be attached to the mobile remote alcohol monitoring device, for example to a hand grip to be held or to a button to be pushed by the monitored person when blowing into the straw.

### BRIEF DESCRIPTION OF THE DRAWINGS

The present disclosed subject matter may be understood and appreciated more fully through the following detailed description taken in conjunction with the drawings, in which corresponding or like numerals or characters indicate corresponding or like components. However, the use of a number to refer to a component in a given figure is not intended to limit the component in another figure labeled with the same number. Unless indicated otherwise, the drawings provide exemplary embodiments or aspects of the present disclosure and do not limit its scope. In the drawings:

FIG. 1 shows a schematic drawing of a system for using the mobile remote alcohol monitoring device, in accordance with some exemplary embodiments of the present disclosure;

FIG. 2 shows a schematic block diagram of the mobile remote alcohol monitoring device, in accordance with some exemplary embodiments of the present disclosure;

FIG. 3 shows a more detailed schematic block diagram of the mobile remote alcohol monitoring device, in accordance with some exemplary embodiments of the present disclosure;

FIG. 4 shows a schematic drawing of the ECG circuit, in accordance with some exemplary embodiments of the present disclosure;

FIG. 5 shows a flowchart diagram of a method for using a mobile remote alcohol monitoring device, in accordance with some exemplary embodiments of the present disclosure; and

FIG. 6 shows a schematic drawing of the mobile remote alcohol monitoring device including an ECG sensor for identifying the monitored person using the mobile remote alcohol monitoring device, in accordance with some exemplary embodiments of the present disclosure.

### DETAILED DESCRIPTION

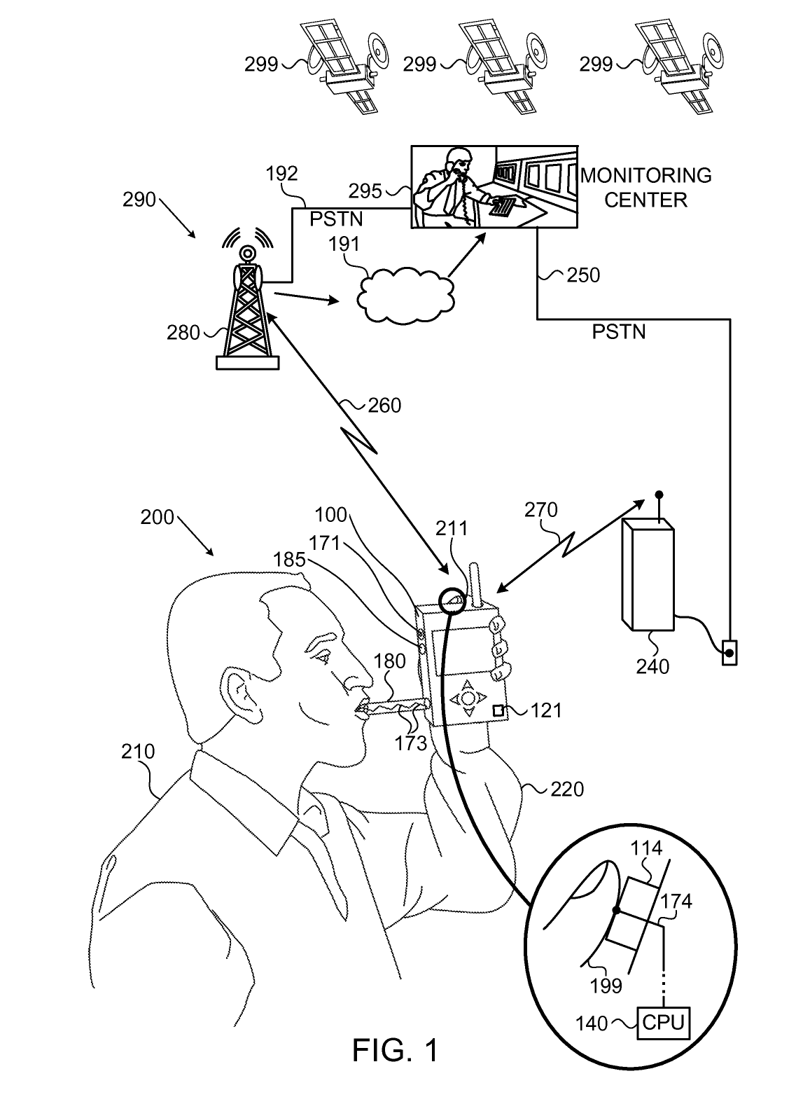

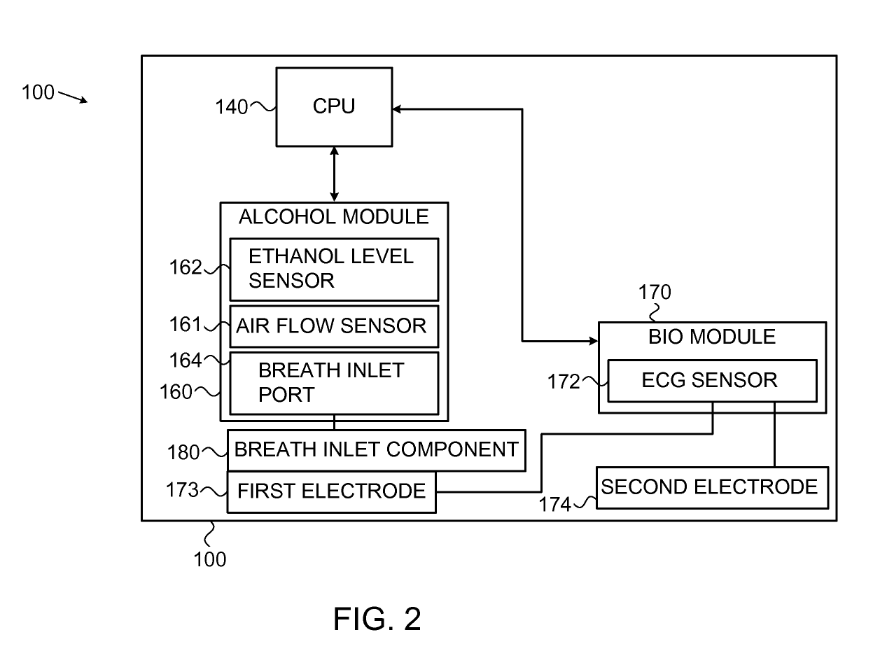

FIG. 1 shows a schematic drawing of a system for using the mobile remote alcohol monitoring device 100, in accordance with some exemplary embodiments of the present disclosure. In FIG. 1, a monitored person 210 is holding a mobile remote alcohol monitoring device 100. The mobile remote alcohol monitoring device 100 is designed to take a breath test from the monitored person 210 to estimate the monitored person's 210 blood alcohol level. The monitored person 210 holding the mobile remote alcohol monitoring device 100 is blowing into a breath inlet component 180. Breath inlet component 180 can be a disposable or multiple use conduit of air such as a straw, tube, mask or other device. Breath inlet component 180 is connected to a breath inlet port (164 of FIG. 2) on the mobile remote alcohol monitoring device 100 such that when the monitored person 210 blows into the breath inlet component 180, air passes through the breath inlet component 180 and into the breath inlet for analysis. In some embodiments, the breath inlet component 180 can have a first electrode 173 connected thereto. A second electrode 174 is connected to button 114. Both the first electrode 173 and the second electrode 174 are connected to CPU 140 to form an electrical circuit enabling the capturing of ECG signals.

The mobile remote alcohol monitoring device 100 may also verify the identity of the monitored person 210 while the monitored person 210 is holding the mobile remote alcohol monitoring device 100 and blowing breath into the breath inlet component 180. In some embodiments, the verification is based on an image of the monitored person 210 taken by the mobile remote alcohol monitoring device 100 by an image capturing device 171 such as a camera. In some other embodiments the verification is done by other biometric devices such as a fingerprint capturing device, an ECG sensor, or an iris recognition device, any of which may be attached to the mobile remote alcohol monitoring device 100. While a monitored person 210 may attempt to evade verification, for the purposes of the present disclosure, it is assumed that if an image taken by image capturing device 171 matches a reference image, there is a high likelihood that the identity of the monitored person 210 is verified. A parallel assumption is made with respect to other biometric devices, such as a fingerprint capturing device, ECG sensor or iris recognition device.

In accordance with some exemplary embodiments of the present disclosure, the fingerprint capturing device is embedded within the button 114. In accordance with this exemplary embodiment, the monitored person's 210 fingerprint will be taken during an enrollment process and stored for reference purposes. Later, when the monitored person 210 presses the button 114 with finger 199, the monitored person's 210 fingerprint may be taken and compared with the stored reference fingerprint to verify the identity of monitored person 210. Either finger may be used to press the button 114 depending whether the person is left handed or right handed.

While in contact with the button 114, and the breath inlet component 180 the monitored person's 210 ECG may be captured since an electrical circuit is formed using the first electrode 173 and the second electrode 174. Capturing of ECG is performed through the process of an ECG test. An ECG test is a test that captures the electrical activity of the heart 402. The ECG test is used to measure the rate and regularity of the monitored person's 210 heartbeats as well as the size and position of the chambers of the heart 402. The ECG test also measures any damage to the heart 402. The results of the ECG test vary from one person to another. Thus, each person has his own unique ECG results. In the present disclosure it records the electrical activity of the heart 402 of the monitored person 210. The ECG test includes the recording and amplifying of the electrical changes on the skin of the limb of the monitored person 210 and on the lips of the monitored person 210. These electrical changes are caused when the heart muscle of the monitored person 210 depolarize during each heartbeat. Depolarization is the change in the heart cells' membranes electrical potential making the cells' membranes more negative or more positive. Following the capture of the ECG signals an ECG result is obtained. Each ECG result is unique to one person. Therefore the difference between various ECG results suggests, to a statistical degree, that the ECG results come from various people. Likewise, if the ECG result is different than the reference ECG signals then to a statistical degree the monitored person 210 taking the ECG test is not the same as the person who took the ECG test which resulted in the capturing of the reference ECG signals.

In accordance with some embodiments of the present disclosure, the ECG signal may be captured before, during or after an alcohol breath test is taken. If ECG signals are captured and compared with the reference ECG signals the mobile remote alcohol monitoring device 100 through CPU 140 may determine, to a statistical degree, if the person providing the ECG signals captured is the same person that provided the reference ECG signals stored within the mobile remote alcohol monitoring device 100.

In other embodiments, the verification is done by an iris recognition device, which may be attached to the mobile remote alcohol monitoring device 100 near the same location as or instead of the image capturing device 171. A captured image and/or any other additional biometric information captured is analyzed to determine the likelihood that the monitored person 210 holding the mobile remote alcohol monitoring device 100 is the monitored person 210.

The mobile remote alcohol monitoring device 100 further includes an actuating device 185 mounted on the mobile remote alcohol monitoring 100 device to assist the monitored person 210 to position her or his face while blowing into the breath inlet component 180 such that his or her face is facing the camera. Such actuating device 185 can be for example a small mirror, through which the monitored person 210 can view his face when blowing into breath inlet component 180. The image capturing device 171 can be positioned such that it captures the image the monitored person 210 sees in the small mirror. Actuating device 185 can help ensure an image captured by image capturing device 171 is properly focused, centered, with consistent scale and preferably devoid of background.

The actuating device 185 may be configured to further instruct the monitored person 210 what steps to take to accomplish a biometric test to be applied by the mobile remote alcohol monitoring device 100. For example, actuating device 185 can be a loudspeaker (not shown) from which the monitored person 210 may receive instructions on how to position his face or hand so as to enable the mobile remote alcohol monitoring device 100 to acquire a biometric sample.

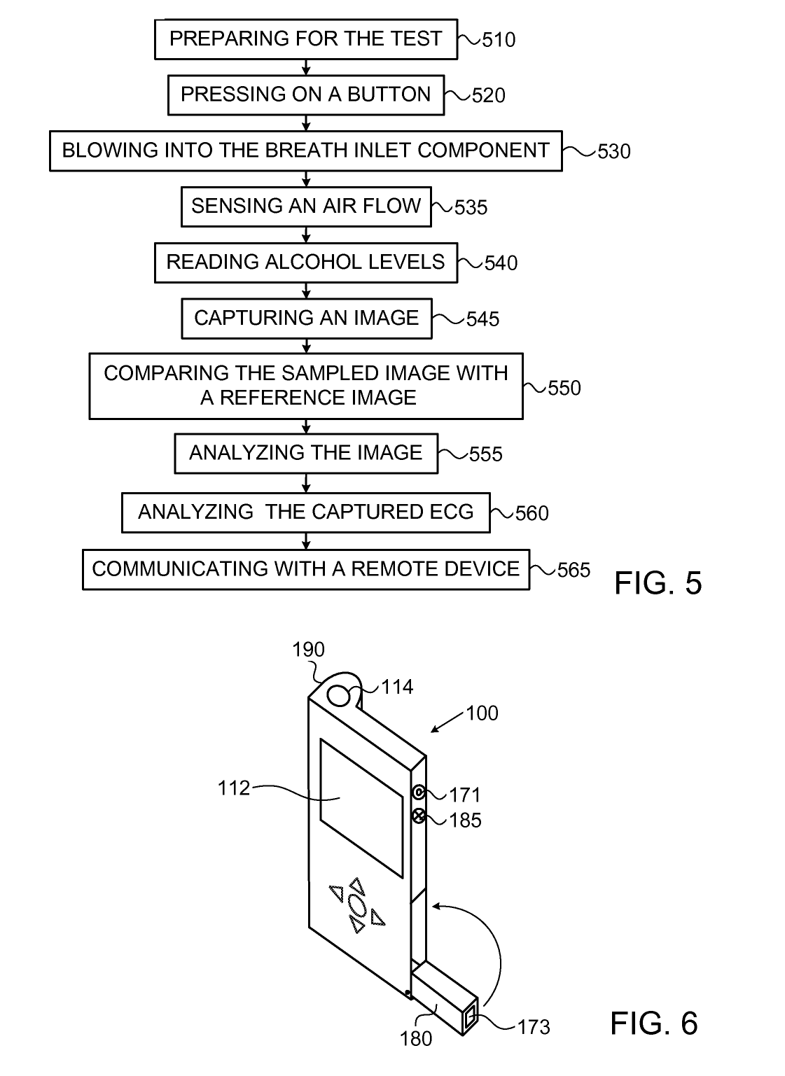

In accordance with some other embodiments, identity verification is based on a biometric or a biological sample of the monitored person 210, such as an ECG signal of the monitored person 210 taken by a biological capturing device (not shown) in the mobile remote alcohol monitoring device 100 while the monitored person 210 is blowing into the breath inlet component 180. The biological capturing device can record ECG signals of the monitored person 210 by receiving signals from at least two electrodes in contact with the monitored person's 210 skin. The first electrode 173 is connected to breath inlet component 180, and the second electrode 174 is connected the grip 190 of the mobile remote alcohol monitoring device 100, a button 114, or another part of the mobile remote alcohol monitoring device 100 that the monitored person 210 is required to press or hold while holding the mobile remote alcohol monitoring device 100 while blowing into breath inlet component 180. In one embodiment, the monitored person 210 contacts the second electrode 174 with a left limb, such as left hand 220 so that an electrical signal generated by the biological capturing device passes through monitored person's 210 heart 402, as discussed in further detail with respect to FIG. 5. The signals taken by the first electrode 173 and the second electrodes (not shown) are received by the biological capturing device for analysis, so as to determine the likelihood that the monitored person 210 holding the mobile remote alcohol monitoring device 100 is the monitored person 210.

In some embodiments, both the image capturing device 171 and the biological capturing device are used to provide a greater certainty with respect to the identity of the monitored person 210 such that there is a better likelihood that the monitored person 210 is both holding the mobile remote alcohol monitoring device 100 and blowing air into the breath inlet component 180.

Other biometric devices, such as an iris capturing device or a fingerprint capturing device can also be used in combination with the image capturing device 171 and the biological capturing device.

In some embodiments of the present disclosure, the image or signals captured may be analyzed by the mobile remote alcohol monitoring device 100 or may be sent to a monitoring center 295 for remote analysis. In some embodiments, an area monitoring device 240 may be located at the monitored person's home (not shown) or other locations in the vicinity of where the monitored person 210 is located. An area monitoring device 240 can be configured to identify various mobile remote alcohol monitoring devices 100 or other mobile devices in the vicinity of the area monitoring device 240. The area monitoring device 240 may receive signals from various mobile devices and may communicate information or data to the monitoring center 295. In such embodiments, when the monitored person 210 is in the vicinity of the area monitoring device 240, the mobile remote alcohol monitoring device 100 and the area monitoring device 240 may establish a communication link and subsequently either one or all of the images, the reference ECG signals, the captured ECG signals captured, the result of any analysis made, and any other relevant information or data may be transferred via a wireless signal 270 to the area monitoring device 240. Information may be transferred periodically or as a result of an event. Such an event may occur when the monitored person's 210 blood alcohol level exceeds a threshold. Communication between the area monitoring device 240 and the monitoring center 295 may occur via Public Switch Telephone Network, referred to as PSTN 250 or Internet Protocol (IP) communication over Cable or Asymmetric Digital Subscriber Line (ADSL) over landline phone. Wireless signal 270 may be an RF signal.

In some other embodiments, the image, ECG signals, other signals captured, the result of any analysis made, and any other relevant information or data may be transferred via a wireless connection 260 to a wireless antenna 280 and from the wireless antenna 280 to the monitoring center 295 also via a wireless network 191, IP over cables or ADSL. The wireless network 191 may be any telephone or data network, including for example the Internet. In some other embodiments, mobile remote alcohol monitoring device 100 further includes a global navigation satellite system (GNSS) unit 121. Through the GNSS satellites 299, the GNSS unit 121 determines the spatial location of the monitored person 210. In some exemplary embodiments, the GNSS unit 121 may be a GPS.

In some embodiments, the analysis of the image, data, or signals captured may be performed by the area monitoring device 240. The results of any such analysis may be sent to the mobile remote alcohol monitoring device 100 or the monitoring center 295. The monitoring center 295 may display the results, or may issue an alarm if the results exceed a threshold. Such an alarm can be sent to law enforcement agencies, police, supervising agencies, and any other person defined by the monitoring center 295.

FIG. 2 shows a schematic block diagram of the mobile remote alcohol monitoring device 100, in accordance with some exemplary embodiments of the subject matter. Mobile remote alcohol monitoring device 100 may include a CPU 140 for controlling the operation of the mobile remote alcohol monitoring device 100. The mobile remote alcohol monitoring device 100 may also include a breath alcohol module 160 for receiving, testing, and analyzing the breath flowing through the breath inlet component 180 into breath inlet port 164. In some exemplary embodiments of the present disclosure the breath inlet component 180 may be a straw or a tube connected to the breath inlet port 164 through which air flows from the mouth of the monitored person 210 into the breath alcohol module 160. In other exemplary embodiments of the present disclosure the breath inlet component 180 may be a mask placed on the face of the monitored person 210, said mask is connected to the breath inlet port 164. In some embodiments, the breath alcohol analysis is performed by CPU 140 or in a remote computerized device as further explained in detail in association with FIG. 1.

In some embodiments, the mobile remote alcohol monitoring device 100 further includes a biological capturing module 170 for obtaining a biometric or biological sample of the monitored person 210 (of FIG. 1). In some embodiments, the biological capturing module 170 records ECG signals. In some embodiments of the present invention, the biological capturing module 170 analyzes the ECG signals captured. In some embodiments of the present invention, the CPU 140 analyzes the sample ECG signals captured. In some other embodiments the biometric or biological sample is analyzed in a remote computerized device as further explained in detail in association with FIG. 1.

The breath alcohol module 160 may include a breath inlet port 164 for receiving the breath flowing through breath inlet component 180, an air flow sensor 161 for sensing the pressure from the air flow and for identifying that a sufficient breath pressure was received through the breath inlet port 164 and an ethanol level sensor 162 for measuring the alcohol level in the breath. The results of the alcohol level in breath may be used to estimate the blood alcohol content (BAC). When the monitored person 210 exhales into the mobile remote alcohol monitoring device 100 any ethanol present in the breath is oxidized to acetic acid at the anode of the ethanol level sensor 162 and at the cathode of the ethanol level sensor 162 atmospheric oxygen is reduced, with an overall reaction of oxidation of ethanol into acetic acid and water. This reaction produces an electrical current which is measured by the ethanol level sensor 162 and sent by the breath alcohol module 160 to the CPU 140. The breath alcohol module 160 is electrically connected to and controlled by central processing unit, referred to as CPU 140. The breath alcohol module 160 may also be connected to the CPU 140 for providing alcohol level results or any other information or data that are read or produced by the ethanol level sensor 162 to CPU 140. When the air flow pressure exceeds a certain threshold, the air flow sensor 161 may signal the CPU 140. The CPU 140 may then activate the ethanol level sensor 162 or biological capturing module 170.

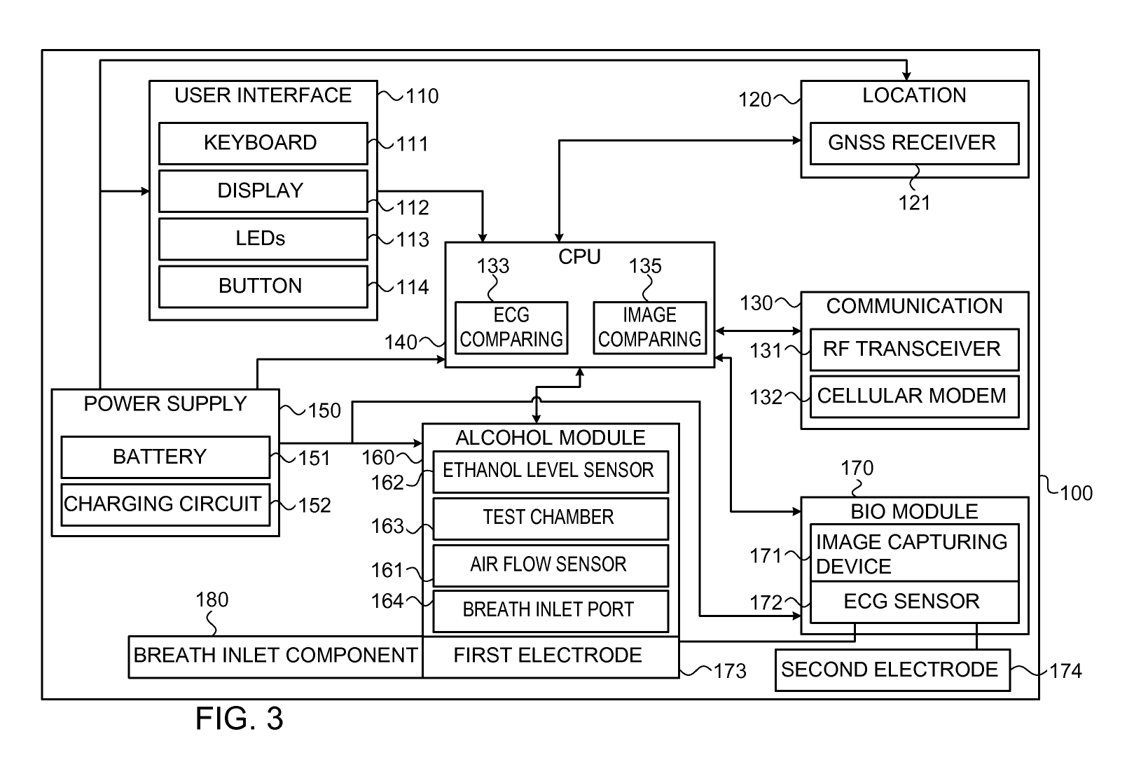

In some embodiments, when a monitored person 210 is required to provide a breath sample to be tested for alcohol level, the CPU 140 may actuate a signal, for example through presenting a message on a screen or providing an audio message, to the monitored person 210 to blow into the breath inlet component 180. In some embodiments of the present disclosure, the CPU 140 provides such signals to the monitored person 210 at predefined times or at random intervals or at the request of a third party, such as for example, a law enforcement officer. According to this embodiment, the CPU 140 will next wait to receive an indication that the button 114 was pressed and an indication from the air flow sensor 161 that air has begun to flow through the breath inlet component 180. An indication that the button 114 was pressed is delivered through closing of an electrical circuit when the button 114 is pushed. After waiting a predetermined period of time after airflow has begun, the CPU 140 will command the ethanol level sensor 162 to take an air sample and measure the alcohol level in the breath. In some other embodiments, the air flow sensor 161 is directly connected to the ethanol level sensor 162. In some embodiments, when air flow sensor 161 is actuated by the breath flow, the air flow sensor 161 actuates the ethanol level sensor 162 to measure ethanol levels in the breath. Ethanol level sensor 162 can be actuated, for example, according to predetermined rules programmed into the mobile remote alcohol monitoring device 100. Such rules can include, for example, actuation of the ethanol level sensor 162 at regular intervals, such as every 30 seconds or every 30 minutes. Other predetermined rules may include time of the day, dates, external factors, such as location, biometric test results, messages received, or messages sent, image captured by the image capturing device (171 of FIG. 3) and the like.

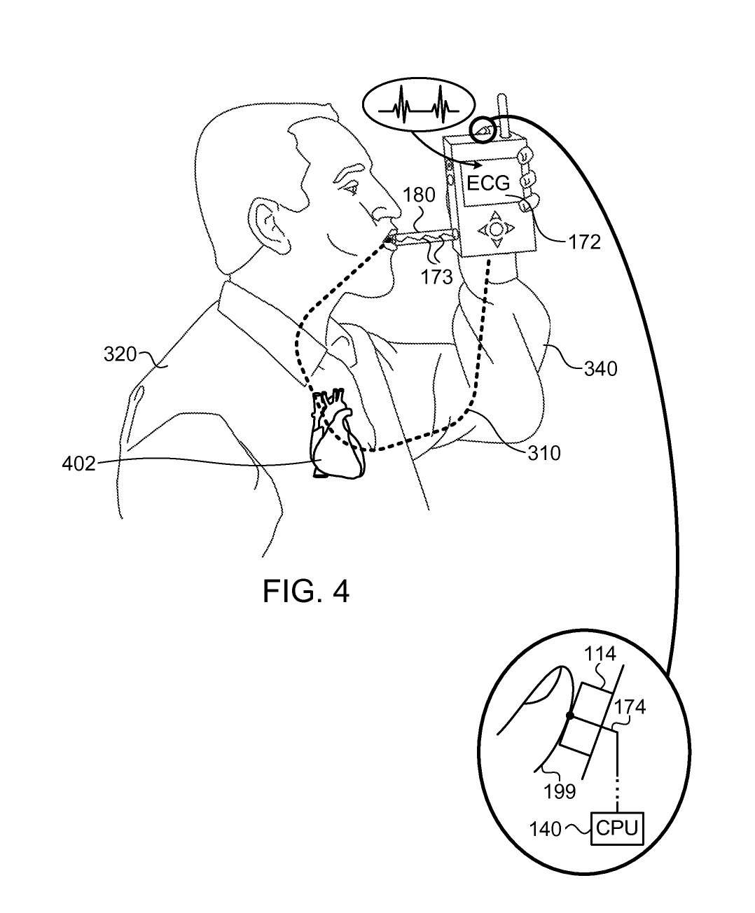

The biological capturing module 170 may include an ECG sensor 172. The ECG sensor 172 may be a miniaturized chip-set, which may run on the mobile remote alcohol monitoring device 100 microprocessor. The ECG sensor 172 may include a sensor integrated circuit (not shown). The sensor integrated circuit (not shown) may utilize a bio-amplifier, bio-filters and A/D converter in order to acquire high quality electro physiological signals from the monitored person 210 touching the contact points. One example of such an ECG sensor 172 is the BDS™ Sensor Chipset, manufactured by IDesia of Israel. The ECG sensor 172 is connected to at least two electrodes, such as the first electrode 173 and the second electrode 174. During the alcohol breath test the monitored person 210 (of FIG. 1) is in contact with the first electrode 173 and the second electrode 174 to form a closed electrical circuit. When the electrical circuit is closed the ECG sensor 172 performs an ECG test by capturing the ECG signals of the monitored persons' (210) heart 402 of FIG. 4. In some embodiments, the first electrode 173 is placed on the breath inlet component 180 such that when the monitored person 210 is in contact with the breath inlet component 180 his lips or tongue also touch the first electrode 173. In some embodiments the second electrode 174 is located on the grip (211 of FIG. 1) or the button (114 of FIG. 6) of the mobile remote alcohol monitoring device 100 such that when an alcohol breath test is taken, the monitored person (210 of FIG. 1) holding the mobile remote alcohol monitoring device 100 touches or is in contact with the second electrode 174. In some embodiments, the monitored person (210 of FIG. 1) is required to press the button (114 of FIG. 6) when an alcohol breath test is taken.

The ECG sensor 172 may take a reference ECG signal when the monitored person 210 is initially registered to the mobile remote alcohol monitoring device 100. This reference ECG signal may be electronically stored in a storage (not shown) and may be used for analysis or comparison with sample ECG signal taken when testing the breath alcohol level of the monitored person 210. The reference sample signal may be stored in the monitoring center 295, or in other devices as further described above in connection with FIG. 1. The analysis of the sample signal and the reference sample signal may be performed for authenticating the identity of monitored person 210 (of FIG. 1). In one embodiment, the CPU 140 may include an ECG analysis unit (not shown) for analyzing the sampled signals with a reference sample previously taken or stored.

The monitored person (210 of FIG. 1) may be required to press a button (114 of FIG. 6) to activate an alcohol breath test. In some preferred embodiments, the mobile remote alcohol monitoring device 100 maintains a low power consumption mode to save on battery life. In a low power consumption mode, the mobile remote alcohol monitoring device 100 may be set to a default OFF mode. In this exemplary embodiment, when pressed, button 114 may wake or switch the mobile remote alcohol monitoring device 100 to an ON mode. When pressed, button 114 may actuate the CPU 140 and in return the CPU 140 powers up the breath alcohol module 160, which is preferably maintain powered down to conserve power. The breath inlet component 180 may be used by the monitored person 210 for directing the breath of the monitored person 210 (of FIG. 1) into the breath inlet port 164. In accordance with some exemplary embodiments, the breath inlet component 180 is a cylindrical tube, such as a straw, made of disposable material. One end of the breath inlet component 180 can be attached to the mobile remote alcohol monitoring device 100. The breath inlet component 180 may include the first electrode 173 for closing the electrical circuit for capturing ECG signals, and in some embodiments, for activating the air flow sensor 161. In some other embodiments of the present disclosure, the breath inlet component 180 may take different shapes and sizes.

In some embodiments of the present disclosure, the air flow sensor 161 senses the pressure of the air moving through the breath inlet component 180. After a predetermined time, such as for example a few seconds, the CPU 140 commands the breath alcohol module 160 to sample the air moving through the breath inlet component 180. Such wait is preferred so that the deep lung air of the monitored person 210 is captured and tested. The ethanol level sensor 162 of the breath alcohol module 160 captures a sample of the air, analyzes the alcohol level in the sample captured, and provides a result to the breath alcohol module 160 or directly to CPU 140.

In some other embodiments of the present disclosure, the CPU 140 may measure the time beginning at the start of a blow, or beginning when the air flow sensor 161 reaches an air pressure threshold, and command the ECG sensor 172 to start sampling the breath received after a predetermined amount of time has passed. In accordance with some exemplary embodiments of the present disclosure, the designated amount of time includes sufficient time for the deep air in the lungs to be sampled, such as for example over 5 seconds. The designated amount of time may be predetermined by a person setting the configuration of the mobile remote alcohol monitoring device 100 or may be determined by a person at the monitoring center (295 of FIG. 1) and transmitted to the mobile remote alcohol monitoring device 100.

FIG. 3 shows a more detailed schematic block diagram of the mobile remote alcohol monitoring device, in accordance with some exemplary embodiments of the subject matter. The mobile remote alcohol monitoring device 100 may generally include CPU 140 for controlling the operation of the mobile remote alcohol monitoring device 100. The mobile remote alcohol monitoring device 100 may also include breath alcohol module 160 for monitoring the alcohol levels of the monitored person 210 and biological capturing module 170 for verifying the identity of the monitored person 210 (of FIG. 1). Communication device 130 can facilitate communication with a remote device, for example the monitoring center 295 (of FIG. 1), and user interface unit 110 allows communication with the monitored person 210 and enables the monitored person 210 to activate some of the features of the mobile remote alcohol monitoring device 100. Location unit 120 provides location information to the mobile remote alcohol monitoring device 100. Mobile remote alcohol monitoring device 100 also includes breath inlet component 180 for directing the breath of the monitored person 210 to the breath inlet port 164, and power supply unit 150.

The breath alcohol module 160 may also include, in addition to the modules that are disclosed in FIG. 2, a test chamber 163 for capturing an air sample, typically one or more seconds after the start of the blow.

The biological capturing module 170 may include, in addition to the modules that are disclosed in FIG. 2, an image capturing device 171, such as a camera, a biological or biometric capturing device, such as an ECG sensor 172 or other bio verification devices such as, for example, voice detection device, fingerprint detection device or a combination thereof. The image capturing device 171 may be configured for capturing one or more reference images of the monitored person 210. Reference images can be used to later authenticate the identity of the monitored person 210 while the monitored person 210 is blowing into the breath inlet component 180 and holding the mobile remote alcohol monitoring device 100. The identity of the monitored person 210 can be authenticated by comparing the captured image with the reference image for analyzing the captured image. Comparing may be performed by an image comparing device 135 within the mobile remote alcohol monitoring device 100 or by a remotely located device, such as the monitoring center 295. In some embodiments, such a comparison may be performed within a short time after the image of monitored person 210 is captured.

The communication device 130 may include a wireless communication modem such as cellular modem 132 and RF transceiver 131. The communication device 130 may be used for communicating with a remote device (not shown). Communication may be, for example, for transmitting sampling results, including a captured image or a sample ECG signal, triggering alarms regarding alcohol levels and/or regarding location of the monitored person 210. The alarms based on the alcohol levels may be trigger if the alcohol levels exceed a threshold. The communication device 130 may also be used for verbal communication between the monitored person 210 and an officer.

The location unit 120 may include a GNSS receiver, referred to as GNSS unit 121. The GNSS unit 121 may include a GPS receiver. The location unit 120 may be used for locating the monitored person 210. The location unit 120 may periodically sample the location of the monitored person 210. The mobile remote alcohol monitoring device 100 may store a list of locations that are forbidden for the monitored person 210. Such locations may be, for example, a pub and the like. When the monitored person 210 arrives at a forbidden area, the mobile remote alcohol monitoring device 100 may notify a remote station via the communication device 130.

The user interface unit 110 may include a keyboard 111, a display 112, one or more LEDs 113 and a button 114. The display 112 may be an LCD display. The keyboard 111 may be used for enabling the monitored person 210 to operate the mobile remote alcohol monitoring device 100 and for writing messages to send to a remote device. The LEDs 113 may be used for indicating statuses of the mobile remote alcohol monitoring device 100. For example if one unit of the mobile remote alcohol monitoring device 100 does not work, an LED may be turned on. Pressing the button 114 may close an electrical circuit between the image capturing device 171 and/or to the ECG sensor 172 or any other verification device and the air flow sensor 161.

The CPU 140 may measure the time since the start of the blow of air into the breath inlet component 180 as indicated by the air flow sensor 161 and command the ECG sensor 172 to begin capturing ECG signals from the monitored person 210 and the image capturing device 171 to capture an image of the monitored person 210 holding the mobile remote alcohol monitoring 100. The CPU 140 may include an ECG comparing device 133 for comparing the captured ECG signals with a reference ECG signal previously stored on the mobile remote alcohol monitoring 100 or stored in a remote location, such as for example, the area monitoring device 240 or the monitoring center 295. The CPU 140 may include an image comparing device 135 for comparing the image captured by the image capturing device 171 with a reference image previously stored on the mobile remote alcohol monitoring 100 or stored in a remote location, such as for example, the area monitoring device 240 or the monitoring center 295.

The power supply unit 150 may include a rechargeable battery 151 and a charging circuit 152 for charging the rechargeable battery 151.

FIG. 4 shows a schematic drawing of the ECG circuit, in accordance with some exemplary embodiments of the subject matter. When a monitored person 320 performs an alcohol breath test by blowing into breath inlet component 180, a closed electrical circuit is generated between the first electrode 173 and the second electrode 174. The first electrode 173 is in contact with the lips of the monitored person 320, and the second electrode 174 is on a button 114 pressed by the monitored person's 320 left hand 340 finger 199. The second electrode 174 can also be attached to a grip 190 or other portion of the mobile remote alcohol monitoring device 100 that will come into contact with one of the monitored person's 210 limbs. The first electrode 173 may be attached to the breath inlet component 180. The first electrode 173 and the second electrode 174 may be electrically connected to the ECG sensor 172.

FIG. 5 shows a flowchart diagram of an exemplary method for using a mobile remote alcohol monitoring device, in accordance with some exemplary embodiments of the present disclosure. In step 510, the monitored person 210 may prepare for a breath alcohol level test employing the mobile remote alcohol monitoring device 100. The preparation for the test may include the monitored person 210 contacting the breath inlet component 180 forming contact with the first electrode 173 and the second electrode 174. In some embodiments, the preparation may also include positioning the monitored person's 210 head such that his face is facing the image capturing device 171, preferably resulting in a properly focused, centered identifying image of consistent scale and preferably devoid of background of the monitored person.

In step 520, the monitored person may press on a button, such as button 114 of FIG. 6. In some embodiments, the second electrode 174 may be located on the button 114, thus when the button 114 is pressed the monitored person is in electrical contact with the second electrode 174. The button 114 may be used for activating an alcohol breath test by the monitored person. When pressed, the button 114 may actuate the CPU 140, and in return, the CPU 140 may power up the breath alcohol module 160. Pressing the button 114 may also close an electrical circuit between the first electrode 173 and the second electrode 174, enabling the ECG sensor 172 to capture ECG signals of the monitored persons' 210 heart 402.

In step 530, the monitored person may blow into the breath inlet component 180 to provide a breath sample for testing the alcohol level in the breath. Blowing may be performed while pressing on the button 114. Blowing may be performed after an indication is provided to the monitored person 210 to begin blowing air into the breath inlet component 180. Such indication may be auditory or visual.

In step 535 the airflow sensor is sensing the air pressure of the air moving through the breath inlet component 180. The air flow sensor 161 may indicate to the breath alcohol module 160 that air pressure was detected. The breath alcohol module 160 or the CPU 140 may instruct the ethanol level sensor 162 to measure ethanol level in the air received through the breath inlet port 164.

In step 540 the alcohol level of the in the air sample received through the breath inlet port 164 may be measured. The ethanol level sensor 162 determines the level of ethanol in the air received and determines the alcohol level in the air. The breath alcohol module 160 may provide the alcohol level result to the CPU 140. The CPU 140 may determine if the alcohol level exceeds a predetermined threshold, and may issue an alarm. The alarm may be sent to a remote monitoring device, such as the area monitoring device 240 or the monitoring center 295. The CPU 140 may send the alcohol level result to a remote monitoring device, such as the area monitoring device 240 or the monitoring center 295. The CPU 140 may store the alcohol level result.

In step 545, an image of the monitored person 210 may be captured. The image is preferably captured while the monitored person is blowing into the breath inlet component 180.

In step 550, the image that was captured in step 545 may be analyzed. The analysis may be performed by image comparing unit 135. Analyzing may include comparing the image captured of the monitored person 210 with a reference image to determine the likelihood that the person holding the mobile remote alcohol monitoring device 100 is the monitored person 210. The analyzing may be performed by comparing the captured image and the reference image for differences. In such an exemplary method, the two images are first aligned so that corresponding points coincide, and their photometric values are compatible. Alignment may also be done by post-processing methods such as color mapping, image recognition, face recognition, statistical similarity, for example. In some embodiments, after aligning the reference image and the image captured by the mobile remote alcohol monitoring device 100, the difference between the two images is calculated by finding the difference between each two corresponding pixels in each image and, summing the difference values comparing the summed value to a pre-defined threshold for determining if the two images identify the same person.

In some embodiments, the analyzing may be performed by the mobile remote alcohol monitoring device 100, in some other embodiments the analyzing may be performed by the area monitoring device 240 or the monitoring center 295. In such embodiments the captured image may be sent to the remote location where the images are analyzed. In such embodiments the reference image may be sent to the remote where the images are analyzed.

In step 560, the electrical activity captured during the captured ECG signals and the reference ECG signals may be analyzed. Analyzing may be performed by the ECG comparing device 133. Analyzing may include comparing the captured ECG signals with the reference ECG signals for identifying if mobile remote alcohol monitoring device 100 is the monitored person 210. Comparing the captured ECG signals with the reference ECG signals may be performed by first aligning the PQRST points. The PQRST points are typically represented by a graph showing the electrical activity of one complete heartbeat in the ECG, either the captured ECG signals or the reference ECG signals. The PQRST includes the P-wave, the QRS complex and the T-wave. The P-wave describes the atrial contraction. The QRS complex describes the ventricular contraction. The T-wave describes the ventricular de-contraction. The PQRST points for the captured ECG signals shows a graph of the electrical activity of one complete heartbeat for the person holding the mobile remote alcohol monitoring device 100 while the ECG test is taken. The PQRST points for the reference ECG signals shows a graph of the electrical activity of one complete heartbeat for the monitored person 210 previously stored mobile remote alcohol monitoring device 100 or in a remote location. The reference ECG signals may be captured and stored during the enrollment process of the monitored person 210. In this step during the analyzing the two ECG graphs are compared preferably through finding the difference values of the PQRST and then comparing these different values to a threshold, to determine if the two ECG graphs identify the same person.

In some embodiments, the analyzing may be done by the monitoring device, in some other embodiments the sample ECG signal may be sent to a remote location to be analyzed.

FIG. 6 shows a schematic drawing of the mobile remote alcohol monitoring device including an ECG sensor for identifying the monitored person using the mobile remote alcohol monitoring device, in accordance with some exemplary embodiments of the subject matter. Mobile remote alcohol monitoring device 100 may include a grip 190 for holding the mobile remote alcohol monitoring device 100 while blowing. The mobile remote alcohol monitoring device 100 may include a display 112 for displaying messages to the monitored person, an image capturing device 171 for taking a picture of the monitored person while the monitored person is blowing. The mobile remote alcohol monitoring device 100 may also include a breath inlet component 180 for blowing into, wherein first electrode 173 is connected to the breath inlet component 180 for capturing ECG signals while the monitored person is blowing. In some embodiments of the present disclosure the breath inlet component 180 may be foldable and fold from a lateral position as shown in FIG. 6 to a parallel position to be stored within or adjacent to the mobile remote alcohol monitoring device 100. When the breath inlet component 180 is in the a parallel position, it is easier for the monitored person 210 to carry the mobile remote alcohol monitoring device 100 and avoid damage to the breath inlet component 180. In some other embodiments, the breath inlet component is a disposable item and be detached from the mobile remote alcohol monitoring device 100 after use. The mobile remote alcohol monitoring device 100 further includes an actuating device 185 mounted on the mobile remote alcohol monitoring 100 device to assist the monitored person 210 to position her or his face while blowing into the breath inlet component 180 such that his or her face is facing the camera. Such actuating device 185 can be for example a small mirror, through which the monitored person 210 can view his face when blowing into breath inlet component 180. The image capturing device 171 can be positioned such that it captures the image the monitored person 210 sees in the small mirror. Actuating device 185 can help ensure an image captured by image capturing device 171 is properly focused, centered, with consistent scale and preferably devoid of background. In some embodiments of the present disclosure, the actuating device 185 may be configured to further instruct the monitored person 210 what steps to take to accomplish a biometric test to be applied by the mobile remote alcohol monitoring device 100. For example, actuating device 185 can include a loudspeaker from which the monitored person 210 may receive instructions on how to position his face or hand so as to enable the mobile remote alcohol monitoring device 100 to acquire a biometric sample.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of program code, which includes one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all devices or step plus function elements in the claims below are intended to include any structure, material, or act for performing the function in combination with other claimed elements as specifically claimed. The description of the present invention has been presented for purposes of illustration and description, but is not intended to be exhaustive or to limit the invention to the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A monitoring apparatus comprising:
an ethanol level sensor;
an ECG sensor for generating an ECG circuit to capture a sampled ECG signal based on an electrical activity of a heart of a monitored person; and
a breath inlet component; wherein the ECG circuit is generated when the monitored person is in electrical contact with a first electrode of the ECG sensor attached to the breath inlet component and with a second electrode of the ECG sensor.

2. The monitoring apparatus of claim 1, further comprising the first electrode of the ECG sensor and the second electrode of the ECG sensor.

3. The monitoring apparatus of claim 1, further comprising an air flow sensor.

4. The monitoring apparatus according to claim 1, further comprising a global navigation satellite system (GNSS) unit for providing spatial location of the monitoring apparatus.

5. The monitoring apparatus according to claim 1, further comprising a button, wherein the second electrode is attached to the button.

6. The monitoring apparatus according to claim 1, further comprising a grip, wherein the second electrode is attached to the grip.

7. The monitoring apparatus according to claim 1, further comprising an image capturing device for capturing an image of the monitored person.

8. The monitoring apparatus according to claim 7, further comprising an image comparing device for comparing a captured image with a reference image.

9. The monitoring apparatus according to claim 1, further comprising a communication device.

10. The monitoring apparatus according to claim 1, further comprising an ECG comparing device for comparing captured ECG signals with a reference ECG signals.

11. A method of monitoring a person, the method comprising the steps of:
(a) sensing an air flow of a breath sample provided by the monitored person;
(b) reading an ethanol level result from the breath sample;
(c) generating an ECG circuit for capturing a sampled ECG signal by sampling an electrical activity of a heart of the person while the person is providing the breath sample.

12. The method according to claim 11, wherein step (b) comprises reading the ethanol level result from the breath sample when the air flow is above a predetermined level.

13. The method according to claim 11, further comprising the step of:
(d) determining a spatial location of the person.

14. The method of claim 11, further comprising the step of:
(e) capturing an image of the person while sensing the air flow of the breath sample.

15. The method of claim 14, further comprising the step of: (f) comparing the image of the person with a reference image.

16. The method of claim 11, further comprising the step of: (d) comparing the sampled ECG signal with a reference ECG signal.

17. A method of making a monitoring device, the method comprising the steps of:
(a) providing an ethanol level sensor capable of reading an ethanol level result from a breath sample from a monitored person;
(b) providing an ECG sensor capable of generating an ECG circuit for capturing an ECG signal by sampling an electrical activity of a heart of the monitored person;
(c) assembling the ethanol level sensor and the ECG sensor as parts of the monitoring device.

18. The method according to claim 17, further comprising the step of: (d) providing a breath inlet component capable of conducting the breath sample to the ethanol level sensor.

19. The method of claim 17, further comprising the step of: (d) providing an air flow sensor capable of sensing an air flow of the breath sample.

20. The method of claim 17, further comprising the step of: (d) providing an ECG analyzing device capable of comparing captured ECG signals with a reference ECG signals.
